(19) United States
(12) Patent Application Publication
(10) Pub. No.: US 2019/0022207 A1
(43) Pub. Date: Jan. 24, 2019

(54) Compositions and Methods for Treating Acne

(71) Applicant: Cedars-Sinai Medical Center, Los Angeles, CA (US)

(72) Inventors: George LIU, Los Angeles, CA (US); Stacey KOLAR, West Hollywood, CA (US)

(73) Assignee: Cedars-Sinai Medical Center, Los Angeles, CA (US)

(21) Appl. No.: 15/748,593

(22) PCT Filed: Jul. 29, 2016
(86) PCT No.: PCT/US2016/044793

§ 371 Date: Jan. 29, 2018

Related U.S. Application Data
(60) Provisional application No. 62/198,904, filed on Jul. 30, 2015.

(51) Int. Cl. A61K 39/05; A61K 9/00; A61P 17/10; A61K 38/47

(52) U.S. Cl. CPC A61K 39/05; A61K 9/0014; A61K 2039/6025; A61K 38/47; A61P 17/10

(57) ABSTRACT

In various embodiments, the invention relates to a peptide-based vaccine targeting bacterial hyaluronidase. In some embodiments, the invention includes isolated antibodies which have been raised in response to the bacterial hyaluronidase or one or more portions thereof, compositions or vaccines described herein. The invention further relates to kits for using the peptides, compositions, or antibodies described herein. In still further aspects, the invention also relates to methods for using the peptides, compositions, vaccines, or antibodies.

### FIELD OF THE INVENTION

In various embodiments, the invention relates to compositions, methods and kits for treating or preventing acne and related conditions associated with inflammation.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Acne is a skin condition that causes pimples. This includes whiteheads, blackheads, and red, inflamed patches of skin (such as cysts). Acne occurs when tiny pores on the surface of the skin become clogged. Each pore opens to a follicle. A follicle contains a hair and an oil gland. When glands produce too much oil, the pores can become blocked. Dirt, bacteria, and cells build up. The blockage is called a plug or comedone. If the top of the plug is white, it is called a whitehead. If the top of the plug is dark, it is called a blackhead. If the plug breaks open, swelling and red bumps occur. Acne that is deep in skin can cause hard, painful cysts. This is called cystic acne.

Various treatments exist for the treatment of acne. In general, acne treatments work by reducing oil production, speeding up skin cell turnover, fighting bacterial infection, reducing the inflammation, or doing all four. These types of acne treatments include over-the-counter topical treatments, antibiotics, oral contraceptives and cosmetic procedures. Acne lotions may dry up the oil, kill bacteria and promote sloughing of dead skin cells. Over-the-counter (OTC) lotions are generally mild and contain benzoyl peroxide, sulfur, resorcinol, salicylic acid or sulfur as their active ingredient. Antibiotics may cause side effects, such as an upset stomach, dizziness or skin discoloration. These drugs also often increase sun sensitivity of the skin and may reduce the effectiveness of oral contraceptives. For deep cysts, antibiotics may not be enough. Isotretinoin (Amnesteem, Claravis, Sotret) is a powerful medication available for scarring cystic acne or acne that doesn't respond to other treatments. However, isotretinoin has many side effects, such as dry skin, depression, severe stomach pain, and muscle/joint/back pain, and can cause birth defects in babies whose mothers use isotretinoin. Oral contraceptives, including a combination of norgestimate and ethinyl estradiol (Ortho Tri-Cyclen, Previfem, others), can improve acne in women. However, oral contraceptives may cause other side effects, such as headaches, breast tenderness, nausea, and depression. Chemical peels and microdermabrasion may be helpful in controlling acne. These cosmetic procedures, which have traditionally been used to lessen the appearance of fine lines, sun damage, and minor facial scars, are most effective when used in combination with other acne treatments. They may cause temporary, severe redness, scaling and blistering, and long-term discoloration of the skin.

Considering the shortcomings and risks associated with traditional treatments, there is clearly a need in the art for a vaccine for treating, preventing, reducing the likelihood of, and reducing the severity of acne.

### SUMMARY OF THE INVENTION

In accordance with the purposes of this invention, as embodied and broadly described herein, this invention, in one aspect, relates to P. acnes hyaluronidase or peptides of P. acnes hyaluronidase. In another aspect, the invention relates to pharmaceutical (therapeutic) compositions or vaccines comprising P. acnes hyaluronidase (SEQ ID NO: 1) or peptides of P. acnes hyaluronidase or fragments, variants or peptidomimetics of said peptides. In some embodiments, the peptides comprise, consist of or consist essentially of the sequences set forth in SEQ ID NOs: 2-29. In further embodiments, provided herein are antibodies that specifically bind to P. acnes hyaluronidase (SEQ ID NO: 1) or peptides of P. acnes hyaluronidase or fragments, variants or peptidomimetics of said peptides. In some embodiments, the antibodies specifically bind one or more epitopes having sequences set forth in SEQ ID NOs: 2-29. In some embodiments, the pharmaceutical compositions comprise antibodies that bind epitopes having the sequences set forth in any one or more of SEQ ID NOs: 1-29.

The peptides utilized in preparing the pharmaceutical (therapeutic) compositions or vaccines of the invention can include all or a portion of P. acnes hyaluronidase. In some embodiments, the peptides are immunomodulatory. In one embodiment, the peptides are immunostimulatory. In some embodiments, the invention is a pharmaceutical (therapeutic) compositions or vaccine that includes one or more peptides or protein described or referenced herein. In some embodiments, the pharmaceutical (therapeutic) compositions comprise antibodies described herein. The compositions or vaccines may further comprise additional components, including but not limited to, carriers, vehicles (e.g., encapsulated, liposomes), and other immunomodulatory molecules (e.g., adjuvants). Additionally, a DNA vaccine that includes all or one or more portions of the DNA encoding the peptides described herein, including all or one or more portions of P. acnes hyaluronidase, is within the scope of the present invention.

In various embodiments, the invention teaches isolated antibodies which are elicited in response to one or more one or more peptide fragments, peptides, compositions or vaccines described herein.

In certain embodiments, the invention teaches methods for using the peptide fragments, peptides, compositions, vaccines, or antibodies specific for one or more peptides, fragments thereof, or compositions described herein, for treating, inhibiting, reducing the severity of and/or preventing acne in a subject in need thereof. In some embodiments, the invention teaches using monoclonal or polyclonal antibodies, or fragments thereof, which are specific for any peptides, fragments thereof, combinations thereof, and other components of vaccine compositions described herein for diagnostic purposes, or for accomplishing antibody-targeted delivery strategies.

In some embodiments, the invention still further relates to kits for using the peptides or antibodies, which can, for example, be used for diagnostic purposes or as part of a targeted delivery strategy.

Additional aspects of the invention will be set forth in part in the description which follows, and in part will be obvious from the description, or may be learned by practice of the invention. It is to be understood that both the foregoing general description and the following detailed description are exemplary and explanatory only and are not restrictive of the invention.

Provided herein are methods for treating, inhibiting, reducing the likelihood of having, reducing the severity of and/or slowing the progression of a condition in a subject in need thereof. The methods include providing a composition comprising P. acnes hyluronidase or peptides of P. acnes hyluronidase or fragments, derivatives, variants or peptidomimetics thereof and administering a therapeutically effective amount of the composition to the subject, thereby treating, reducing the likelihood of having, reducing the severity of and/or slowing the progression of the condition in the subject.

Also provided herein are methods for treating, inhibiting, reducing the likelihood of having, reducing the severity of and/or slowing the progression of a condition in a subject in need thereof. The methods include providing a composition comprising antibodies that recognize and bind P. acnes hyluronidase or peptides of P. acnes hyluronidase or fragments, derivatives, variants or peptidomimetics thereof and administering a therapeutically effective amount of the composition to the subject, thereby treating, reducing the likelihood of having, reducing the severity of and/or slowing the progression of the condition in the subject.

In some embodiments, the condition is acne, an infection secondary to acne, or an acne-induced condition. In some embodiments, the acne-induced condition is inflammation, an abscess, pain, or combinations thereof.

In one embodiment, the subject is a human. In various embodiments, the human may be subject is a male or female.

In some embodiments, the composition is administered simultaneously with a topical acne treatment, or subsequent to a topical acne treatment, or prior to a topical acne treatment, or any combination thereof.

In exemplary embodiments, the composition comprises any one or more of the peptides having the sequence set forth in SEQ ID NOs: 2-29.

Also provided herein are pharmaceutical compositions comprising one or more peptides having the sequence set forth in SEQ ID NOs.: 2-29 and a pharmaceutically acceptable carrier. The pharmaceutical composition may further comprise an adjuvant.

Further provided herein is a pharmaceutical composition comprising one or more antibodies that specifically binds to an antigen having the sequence set forth in any one or more of SEQ ID NOs. 1-29. In some embodiments, the antibody is a monoclonal antibody. In some embodiments, the antibody is a polyclonal antibody.

Also provided herein is a polynucleotide encoding a fragment of SEQ ID NO. 1. In some embodiments, the fragment comprises the polynucleotide sequence encoding any one of the peptides in SEQ ID NOs.: 2-29. Further provided herein are cDNA molecules encoding the polynucleotides described herein and vectors encoding the cDNA molecules described herein. Also provided herein are host-vector systems comprising the vectors encoding the cDNA molecules, transfected into a compatible host cell. The host cell may be a prokaryotic cell or a eukaryotic cell.

Also provided herein are vaccines comprising polynucleotides encoding the polypeptides set forth in Table 1. Further provided herein are vaccines comprising polypeptides set forth in Table 1.

Also provided herein are kits for treating, reducing the likelihood of having, reducing the severity of and/or slowing the progression of a condition in a subject in need thereof. The kits include compositions comprising P. acnes hyluronidase or peptides of P. acnes hyluronidase or fragments, derivatives, variants or peptidomimetics thereof and instructions for using the composition to treat, inhibit, reduce the likelihood of having, reduce the severity of and/or slow the progression of the condition in the subject. In some embodiments, the condition is acne, an infection secondary to acne, or an acne-induced condition. In some embodiments, the acne-induced condition is inflammation, an abscess, pain, or combinations thereof. In some embodiments, the kits further comprise a topical acne treatment.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 depicts, in accordance with various embodiments of the invention, clinical isolates of P. acnes secrete the enzyme hyaluronidase. Three clinical isolates and one control strain of P. acnes were placed on an agar plate containing hyaluronan and incubated. A zone of clearing is indicative of hyaluronidase activity.

FIG. 2 depicts, in accordance with various embodiments of the invention, P. acnes hyaluronidase is pro-inflammatory. (a) P. acnes WT and hyaluronidase mutant on a HA plate. (b) Digests of HA with supernatants from WT or hyase mutant were used to stimulate BMDM. (c) TNF-α release from skin infected with P. acnes WT and hyaluronidase.

FIG. 3 depicts, in accordance with various embodiments of the invention, the effect of HA digest by P. acnes hyaluronidase on pro-inflammatory cytokine release from mocrophages.

FIG. 4 depicts, in accordance with various embodiments of the invention, the effect of HA digest by P. acnes hyaluronidase on pro-inflammatory cytokine release from human keraticocyte (HaCa T) cell lines.

FIG. 5 depicts, in accordance with various embodiments of the invention, a new mouse model of P. acnes that was generated for therapeutic testing.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, and lowering the chances of a patient developing the disease condition. As non-limiting examples, “beneficial results” or “desired results” may be alleviation of one or more symptom(s), stabilized (i.e., not worsening) state of acne, delay or slowing of P. acnes infection, and amelioration or palliation of symptoms associated with P. acnes infection, or related conditions associated with inflammation.

“Disorders”, diseases”, “conditions” and “disease conditions,” as used herein may include, but are in no way limited to any form of acne, acne associated inflammation, acne associated infection and acne-induced disorders, and related diseases or conditions. Examples include but are not limited to inflammation, secondary infections, scarring, pain, and the like.

As used herein, a “subject” means a human or animal. The animal may be a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. The methods described herein can also be used to treat animals with immune systems that have been modified to resemble the immune system of a human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., acne or an associated condition) or one or more complications related to the condition, and optionally has already undergone treatment for the condition or the one or more complications related to the condition. Alternatively, a subject can be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “functional” when used in conjunction with “equivalent”, “analog”, “derivative” or “variant” or “fragment” refers to an entity or molecule which possess a biological activity that is substantially similar to a biological activity of the entity or molecule of which it is an equivalent, analog, derivative, variant or fragment thereof.

The terms “hyaluronate lyase”, “hyase,” “HYAse,” and “hyaluronidase” are used interchangeably herein.

As used herein, P. acnes refers to the bacterium Propionibacterium acnes.

As used herein, the term “immunotherapy” refers to the treatment of disease via the stimulation, induction, subversion, mimicry, enhancement, augmentation or any other modulation of a subject's immune system to elicit or amplify adaptive or innate immunity (actively or passively) against harmful proteins, cells or tissues. Immunotherapies (i.e., immunotherapeutic agents) include vaccines, immunomodulators, “antibody-based immunotherapies” or monoclonal antibodies (e.g., humanized monoclonal antibodies), immunostimulants, cell-based therapies such as adoptive T-cell therapies or dendritic cell immunotherapies or dendritic cell vaccines, and viral therapies.

A “gene” or “coding sequence” or a sequence which “encodes” a particular protein or peptide is a nucleic acid molecule that is transcribed (in the case of DNA) and translated (in the case of mRNA) into a polypeptide in vitro or in vivo when placed under the control of appropriate regulatory sequences. The boundaries of the gene are determined by a start codon at the 5′ (i.e., amino) terminus and a translation stop codon at the 3′ (i.e., carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present, so long as the selected coding sequence is capable of being replicated, transcribed and translated in an appropriate host cell.

The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

“Gene transfer” or “gene delivery” refers to methods or systems for reliably inserting foreign DNA into host cells. Such methods can result in transient expression of non-integrated transferred DNA, extrachromosomal replication and expression of transferred replicons (e.g., episomes), or integration of transferred genetic material into the genomic DNA of host cells. Gene transfer provides a unique approach for the treatment of acquired and inherited diseases. A number of systems have been developed for gene transfer into mammalian cells. See, e.g., U.S. Pat. No. 5,399,346. Examples of well-known vehicles for gene transfer include adenovirus and recombinant adenovirus (RAd), adeno-associated virus (AAV), herpes simplex virus type 1 (HSV-1), and lentivirus (LV).

“Recombinant virus” refers to a virus that has been genetically altered (e.g., by the addition or insertion of a heterologous nucleic acid construct into the particle).

“Genetically modified cells”, “genetically engineered cells”, or “modified cells” as used herein refer to cells that express the polynucleotide encoding the polypeptide having the sequence of any one or more of SEQ ID NOs.: 1-29 or a combination thereof, or a variant, derivative, pharmaceutical equivalent, peptidomimetic or an analog thereof. In some embodiments, the peptides described herein or variants, derivatives, pharmaceutical equivalents, peptidomimetics or an analog thereof, are conjugated with agents such as cellulose, fatty acids, polyethylene glycol (PEG) or combinations thereof.

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

“Naked DNA” as used herein refers to DNA encoding a CAR cloned in a suitable expression vector in proper orientation for expression. Viral vectors which may be used include but are not limited SIN lentiviral vectors, retroviral vectors, foamy virus vectors, adeno-associated virus (AAV) vectors, hybrid vectors and/or plasmid transposons (for example sleeping beauty transposon system) or integrase based vector systems. Other vectors that may be used in connection with alternate embodiments of the invention will be apparent to those of skill in the art.

“Transduction” as used herein refers to the introduction of a foreign nucleic acid into a cell using a viral vector.

The term “transfection” is used herein to refer to the uptake of foreign DNA by a cell. A cell has been “transfected” when exogenous DNA has been introduced inside the cell membrane. A number of transfection techniques are generally known in the art. See, e.g., Graham et al. Virology, 52:456 (1973); Sambrook et al. Molecular Cloning, a laboratory manual, Cold Spring Harbor Laboratories, New York (1989); Davis et al., Basic Methods in Molecular Biology, Elsevier (1986), and Chu et al. Gene 13:197 (1981). Such techniques can be used to introduce one or more exogenous DNA moieties, such as a plasmid vector and other nucleic acid molecules, into suitable host cells. The term refers to both stable and transient uptake of the genetic material.

“Vector”, “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc.

“Peptidomimetic” as used herein is a small protein-like chain designed to mimic a protein function. They may be modifications of an existing peptide or newly designed to mimic known peptides. They may be, for example peptoids and/or β-peptides and/or D-peptides.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The term “effective amount” as used herein refers to the amount of a pharmaceutical composition comprising one or more peptides as disclosed herein or a mutant, variant, analog or derivative thereof, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment. In one embodiment, the pharmaceutical (therapeutic) composition comprises, consists of or consists essentially of any one or more peptides described in Table 1. In another embodiment, the pharmaceutical composition comprises a vaccine comprising, consisting of or consisting essentially of any one or more peptides described in Table 1. In some embodiments, the pharmaceutical composition (therapeutic) comprises one or more antibodies that specifically bind P. acnes hyaluronidase having the sequence set forth in SEQ ID NO: 1. In some embodiments, the pharmaceutical composition (therapeutic) comprises one or more antibodies specific to epitopes having the sequence set forth in SEQ ID NOs: 2-29. In various embodiments, the pharmaceutical compositions described herein further comprise a pharmaceutically acceptable carrier. In some embodiments, a therapeutic pharmaceutical composition is used, for example, to treat, inhibit, reduce the severity of and/or, reduce duration of acne and/or related symptoms in a subject in need thereof. In some embodiments, the vaccine is used to prevent acne and/or related symptoms in a subject.

“Pharmaceutically acceptable carriers” as used herein refer to conventional pharmaceutically acceptable carriers useful in this invention.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the P. acnes hyluronidase or peptides thereof and/or vaccines thereof as described herein. Measured or measurable parameters include clinically detectable markers of disease, for example, elevated or depressed levels of a biological marker, as well as parameters related to a clinically accepted scale of symptoms or markers for acne. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject

As used herein, “immunomodulatory”, “antigen”, “antigenic”, “immunogen”, “immunogenic” peptides refer to peptides that, when introduced into the body, can modulate (stimulate or inhibit) an immune response. Thus an immunomodulatory/antigenic peptide of P. acnes hyluronidase, or a variant, fragment or peptidomimetic of said peptide refers to peptides of P. acnes hyluronidase or variants, fragments or peptidomimetics thereof that modulate an immune response in the subject.

As used herein, an “immune response” being modulated refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In one embodiment, an immunomodulator stimulates an immune response. In another embodiment, an immunomodulator inhibits an immune response.

“Antibody” as used herein refers to polyclonal antibodies, monoclonal antibodies, humanized antibodies, single-chain antibodies, and fragments thereof such as Fab, F(ab′)₂, Fv, and other fragments which retain the antigen binding function of the parent antibody. In an embodiment, the antibody specifically binds P. acnes hyluronidase or one or more peptides described herein or variants, fragments or peptidomimetics thereof as described herein (for example, in Table 1). The antibody may be polyclonal antibodies, monoclonal antibodies, humanized antibodies, single-chain antibodies, and fragments thereof such as Fab, F(ab′)2, Fv, and other fragments which retain the activity of the parent antibody. The antibody may be a recombinant antibody. The term “recombinant human antibody” can include a human antibody produced using recombinant DNA technology.

“Monoclonal antibody” as used herein refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)2, Fv, and others which retain the antigen binding function of the antibody. Monoclonal antibodies of any mammalian species can be used in this invention. In practice, however, the antibodies will typically be of rabbit or murine origin because of the availability of rabbit or murine cell lines for use in making the required hybrid cell lines or hybridomas to produce monoclonal antibodies. “Human monoclonal antibody” can include a monoclonal antibody with substantially or entirely human CDR amino acid sequences produced, for example by recombinant methods such as production by a phage library, by lymphocytes or by hybridoma cells.

“Humanized antibodies” as used herein means that at least a portion of the framework regions of an immunoglobulin are derived from human immunoglobulin sequences. The term “humanised antibody” can mean an antibody from a non-human species (e.g. mouse) whose protein sequences have been modified to increase their similarity to antibodies produced naturally in humans.

“Single chain antibodies” as used herein refer to antibodies prepared by determining the binding domains (both heavy and light chains) of a binding antibody, and supplying a linking moiety which permits preservation of the binding function. This forms, in essence, a radically abbreviated antibody, having only that part of the variable domain necessary for binding to the antigen. Determination and construction of single chain antibodies are described in U.S. Pat. No. 4,946,778 to Ladner et al.

The term “antigen binding region” can mean a region of the antibody having specific binding affinity for its target antigen, for example, P. acnes hyluronidase or one or more peptides described herein or variants, fragments or peptidomimetics thereof as described herein. The binding region may be a hypervariable CDR or a functional portion thereof. The term “functional portion” of a CDR can mean a sequence within the CDR which shows specific affinity for the target antigen. The functional portion of a CDR may comprise a ligand which specifically binds to P. acnes hyluronidase.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)2; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the light chain variable and constant regions coupled to the heavy chain variable and constant regions, i.e. VL, CL and VH, CH-1.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)2 fragment” can mean a bivalent antigen-binding fragment of a human monoclonal antibody. An F(ab′)2 fragment includes, for example, all or part of the variable regions of two heavy chains- and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide,

Provided herein are pharmaceutical (therapeutic) compositions comprising, consisting of or consisting essentially of P. acnes hyluronidase or one or more peptides described herein (Table 1) or variants, fragments or peptidomimetics thereof and a pharmaceutically acceptable carrier/excipient. Further provided herein, are pharmaceutical (therapeutic) compositions comprising antibodies that bind epitopes having the sequences set forth in any one or more of SEQ ID NOs: 1-29. Also provided herein are vaccines comprising, consisting of or consisting essentially of one or more peptides described in Table 1 and a pharmaceutically acceptable carrier. In various embodiments, the pharmaceutical compositions and vaccines described herein are used in treating, inhibiting, reducing the severity of and/or preventing acne. In some embodiments, the peptides described herein are fused to any one or more of an epitope tag, a half-life extender or a combination thereof. In exemplary embodiments, the pharmaceutical compositions described herein treat, inhibit and/or reduce acne in a subject by 10%, 20%, 30%, 40%, 50%, 60%, 70%, 80%, 90% 95% Or 100%. Also provided herein are antibodies that specifically bind P. acnes hyluronidase as set forth in SEQ ID NO: 1. In some embodiments, the antibodies are specific to the peptides set forth in SEQ ID NO: 2-29 or fragments, variants or peptidomimetics thereof.

In accordance with some embodiments of the present invention, peptides for preparing pharmaceutical (therapeutic) compositions or vaccines comprising, consisting of or consisting essentially of one or more peptides are derived from P. acnes hyluronidase. In one embodiment, P. acnes hyluronidase has the sequence set forth in SEQ ID NO: 1. In some embodiments the peptides for preparing pharmaceutical (therapeutic) compositions comprise, consist of or consist essentially of the sequence set forth in in any one or more of SEQ ID NOs: 2-29 or fragments, variants and/or peptidomimetics thereof. In some embodiments the peptides for preparing vaccines comprise, consist of or consist essentially of the sequence set forth in in any one or more of SEQ ID NOs: 2-29 or fragments, variants and/or peptidomimetics thereof. It is to be noted that the peptide epitopes described herein are provided as exemplars only as the P. acnes hyluronidase protein may vary between isolates, thereby providing multiple variants of P. acnes hyluronidase.

TABLE 1
Full length hyaluronate lyase from
Propionibacterium acnes and peptides thereof.

| | Residue | | SEQ |
|---|---|---|---|
| Name | Range | Sequence | ID NO: |
| Full length | 1-813 | MFGTPSRRTFLTASALSAMALAASPTVTDAIAAPGPDSW | 1 |
| hyaluronate | | SALCERWIDIITGRRAARTSDPRARAIIAKTDRKVAEIL | |
| lyase from | | TDLVSGSSRQTVLISADLRKEQSPFITKTARAIESMACA | |
| Propionibacterium | | WATPGSSYHKDPEILSACIEGLRDFCRLRYNPSQDEYGN | |
| acnes | | WWDWEDGASRAVADVMCILHDVLPPEVMSAAAAGIDHFI | |
| | | PDPWFQQPASVKPTANPVQPVVSTGANRMDLTRAVMCRS | |
| | | IATGDEKRLRHAVDGLPDAWRVTTEGDGFRADGGFIQHS | |
| | | HIPYTGGYGDVLFSGLAMLFPLVSGMRFDIVESARKAFH | |
| | | DQVERGFIPVMYNGQILDDVRGRSISRINESAAMHGIST | |
| | | ARAMLMMADALPTHRAEQWRGIVHGWMARNTFDHLSEPS | |
| | | TLVDISLFDAAAKARPVPESSTPSYFASMDRLVHRTADW | |
| | | LITVSNCSDRIAWYEYGNGENEWASRTSQGMRYLLLPGD | |
| | | MGQYEDGYWATVDYSAPTGTTVDSTPLKRAVGASWAAKT | |
| | | PTNEWSGGLASGSWSAAASHITSQDSALKARRLWVGLKD | |
| | | AMVELTTDVTTDASRAITVVEHRKVASSSTKLLVDGNRV | |
| | | SSATSFQNPRWAHLDGVGGYVFATDTDLSADVATRKGTW | |
| | | IDVNPSRKVKGADEVIERAYASLHVTHHDRPVAWALLPT | |
| | | ASRSHTMALATRPGVEPFTVLRNDATVQAVRSAGALLTK | |
| | | DPTVVTTLAFWKPATCGGVAVNRPALVQTRESANQMEVV | |
| | | IVEPTQKRGSLTVTIEGSWKVKTADSHVDVSCENAAGTL | |
| | | HVDTAGLGGQSVRVTLARQVTQTPSGGGRHDRA | |
| P1: | 10-24 | FLTASALSAMALAASPTVTDAIAAP | 2 |
| P2 | 38-45 | SWSALCER | 3 |
| P34 | 73-94 | KVAEILTDLVSGSSRQTVLISA | 4 |
| P5 | 112-118 | ESMACAW | 5 |
| P67 | 127-147 | KDPEILSACIEGLRDFCRLRY | 6 |
| P89 | 164-218 | ASRAVADVMCILHDVLPPEVMSAAAAGIDHFIPDPWFQQ | 7 |
| | | PASVKPTANPVQPVVS | |
| P10 | 226-234 | LTRAVMCRS | 8 |
| P11 | 243-249 | LRHAVDG | 9 |
| P12 | 269-277 | FIQHSHIPY | 10 |
| P1314 | 281-297 | YGDVLFSGLAMLFPLVSGMRFDIVES | 11 |
| P15 | 316-326 | ERGFIPVMYNG | 12 |
| P16 | 345-353 | AMHGISIAR | 13 |
| P17 | 370-376 | WRGIVHG | 14 |
| P1819 | 383-409 | FDHLSEPSTLVDISLFDAAAKARPVPE | 15 |
| P2021 | 419-439 | MDRLVHRTADWLITVSNCSDR | 16 |
| P22 | 459-466 | GMRYLLLP | 17 |
| P23 | 476-482 | YWATVDY | 18 |
| P24 | 494-501 | PLKRAVGA | 19 |
| P25 | 522-549 | SAAASHITSQDSALKARRLWVGLKDAMV | 20 |
| P26 | 561-582 | RAITVVEHRKVASSSTKLLVDG | 21 |
| P27 | 598-608 | HLDGVGGYVFA | 22 |
| P28 | 635-664 | GADEVIERAYASLHVTHHDRPVAWALLPTA | 23 |
| P29 | 672-686 | LATRPGVEPFTVLRN | 24 |
| P30 | 688-731 | ATVQAVRSAGALLTKDPTVVTTLAFWKPATCGGVAVNRP | 25 |
| | | ALVQT | |
| P31 | 737-745 | QMEVVIVEP | 26 |
| P32 | 750-756 | GSLTVTI | 27 |
| P3334 | 763-784 | KTADSHVDVSCENAAGTLHVDT | 28 |
| P35 | 788-802 | GGQSVRVTLARQVTQ | 29 |

In some aspects, P. acnes hyluronidase or any one or more of the peptides of P. acnes hyluronidase (for example, as set forth in Table 1 or variants, fragments or peptidomimetics thereof) for preparation of pharmaceutical (therapeutic) compositions or vaccines for use in treating, inhibiting, reducing the severity of and/or preventing acne, are a “modified polypeptide” or “modified peptides” comprising non-naturally occurring amino acids. In some aspects, the polypeptides comprise a combination of naturally occurring and non-naturally occurring amino acids, and in some embodiments, the peptides comprise only non-naturally occurring amino acids.

“Modified polypeptides” or “Modified peptide” may include the incorporation of lactam-bridge, head-to-tail cyclization, non-natural amino acids into the peptides (or fragments, derivatives, variants or peptidomimetics thereof) described herein, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Peptide conjugates can therefore be constructed using, for example, D-isomer forms of cell penetrating peptide sequences, L-isomer forms of cleavage sites, and D-isomer forms of therapeutic peptides. Therefore, in some embodiments the peptides as disclosed comprise L and D amino acids, wherein no more than 1, 2, 3, 4, 5, 6, 7, 8, 9, or 10 D-amino acids are included. In certain aspects, the peptides comprise more than 10 D-amino acids, and in certain aspects all the amino acids of the peptides are D-amino acids.

In some aspects, P. acnes hyluronidase or any one or more of the peptides of P. acnes hyluronidase (for example, as set forth in Table 1 or variants, fragments or peptidomimetics thereof) as described herein for use in treating, inhibiting, reducing the severity of and/or preventing acne are “retro-inverso peptide”. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

In some aspects, P. acnes hyluronidase or any one or more of the peptides of P. acnes hyluronidase (for example, as set forth in Table 1 or variants, fragments or peptidomimetics thereof) for preparation of compositions or vaccines for use in treating, inhibiting, reducing the severity of and/or preventing acne are “peptide dendrimers”. Peptide dendrimers are radial or wedge-like branched macromolecules consisting of a peptidyl branching core and/or covalently attached surface functional units. The multimeric nature of these constructs, the unambiguous composition and ease of production make peptide dendrimers well suited for use as protein mimetics, vaccines, multiple antigen peptides (MAPs) and drug and gene delivery vehicles. Some methods commonly used to generate peptide dendrimers include but are not limited to stepwise solid-phase synthesis, chemoselective and orthogonal ligation (Sadler, K and Tam, J. Peptide dendrimers: applications and synthesis. Reviews in Molecular Biotechnology Vol 90, 3-4, May 2002, Pages 195-229).

Other variants of the peptides described herein (for example, as set forth in Table 1 or variants, fragments or peptidomimetics thereof) can comprise conservatively substituted sequences, meaning that one or more amino acid residues of an original peptide are replaced by different residues, and that the conservatively substituted peptide retains a desired biological activity, (for example, modulation of an immune response to treat, inhibit, reduce the severity of and/or prevent acne) that is essentially equivalent to that of the original peptide. Examples of conservative substitutions include substitution of amino acids that do not alter the secondary and/or tertiary structure of protein and peptides described in Table 1, substitutions that do not change the overall or local hydrophobic character, substitutions that do not change the overall or local charge, substitutions by residues of equivalent side-chain size, or substitutions by side-chains with similar reactive groups.

Other examples involve substitution of amino acids that have not been evolutionarily conserved in the parent sequence across species. Advantageously, in some embodiments, these conserved amino acids and structures are not altered when generating conservatively substituted sequences.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics or substitutions of residues with similar side chain volume are well known. Isolated peptides comprising conservative amino acid substitutions can be tested in any one of the assays described herein to confirm that a desired activity, e.g., stimulation of an immune response to treat, inhibit, reduce the severity of and/or prevent acne is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile, Phe, Trp; (2) neutral hydrophilic: Cys, Ser, Thr, Asn, Gln, Ala, Tyr, His, Pro, Gly; (3) acidic: Asp, Glu; (4) basic: His, Lys, Arg; (5) residues that influence chain orientation: Gly, Pro; (6) aromatic: Trp, Tyr, Phe, Pro, His, or hydroxyproline. Non-conservative substitutions will entail exchanging a member of one of these classes for another class.

Particularly preferred conservative substitutions for use in the variants described herein are as follows: Ala into Gly or into Ser; Arg into Lys; Asn into Gln or into His; Asp into Glu or into Asn; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr or into Phe; Tyr into Phe or into Trp; and/or Phe into Val, into Tyr, into Ile or into Leu. In general, conservative substitutions encompass residue exchanges with those of similar physicochemical properties (i.e. substitution of a hydrophobic residue for another hydrophobic amino acid).

Any cysteine residue not involved in maintaining the proper conformation of the isolated peptide as described herein can also be substituted, generally with serine, to improve the oxidative stability of the molecule and prevent aberrant crosslinking. Conversely, cysteine bond(s) can be added to the isolated peptide as described herein to improve its stability or facilitate multimerization.

To enhance stability, bioavailability, and/or delivery of the peptides into the cells, the peptides can be modified. For example, in some embodiments, an isolated peptide as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. The peptides of P. acnes hyluronidase or fragments, variants or peptidomimetics thereof as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof. In some embodiments, the peptides set forth in Table 1 or variants, fragments or peptidomimetics thereof are conjugated with any of cellulose, fatty acids, polyethylene glycol (PEG) or combinations thereof.

In some embodiments, an isolated peptide as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, an isolated peptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, an isolated peptide can be modified, e.g. a moiety can be added to one or more of the amino acids comprising the peptide. In some embodiments, an isolated peptide as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, an isolated peptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties.

In some embodiments, the protein or any one or more of the peptides (for example, as set forth in Table 1 or variants, fragments or peptidomimetics thereof) for preparation of pharmaceutical (therapeutic) compositions or vaccines for use in treating, inhibiting, reducing the severity of and/or preventing acne as described herein can be a fusion peptide or polypeptide. A fusion polypeptide can comprise a peptide linker domain interposed between the first domain of the peptide comprising an amino acid sequence LLF derivatives, variants, functional fragments, prodrug, or analog thereof or the peptides comprising the amino acid sequence SEQ ID NOs: 1-29 or derivatives, variants, functional fragments, prodrug, or analog thereof as described herein and at least a second domain of the fusion peptide. The first peptide domain can be the N-terminal domain or the C-terminal domain or an internal sequence in the case where the partner domain forms after fragment complementation of constituent parts. Methods of synthesizing or producing a fusion protein are well known to those of ordinary skill in the art. The term “fusion protein” as used herein refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. Fusion proteins can include an epitope tag or a half-life extender. Epitope tags include biotin, FLAG tag, c-myc, hemaglutinin, His6, digoxigenin, FITC, Cy3, Cy5, green fluorescent protein, V5 epitope tags, GST, β-galactosidase, AU1, AU5, and avidin. Half-life extenders include Fc domain and serum albumin.

In one aspect, described herein is a vector comprising a nucleic acid encoding the protein or one or more peptides as described herein (Table 1) or variants, fragments or peptidomimetic thereof. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. Many vectors useful for transferring exogenous genes into target mammalian cells are available. The vectors can be episomal, e.g., plasmids, virus derived vectors such cytomegalovirus, adenovirus, etc., or can be integrated into the target cell genome, through homologous recombination or random integration, e.g., retrovirus derived vectors such MMLV, HIV-1, ALV, etc. Many viral vectors are known in the art and can be used as carriers of a nucleic acid modulatory compound into the cell. For example, constructs containing the nucleic acid encoding a polypeptide can be integrated and packaged into non-replicating, defective viral genomes like Adenovirus, Adeno-associated virus (AAV), or Herpes simplex virus (HSV) or others, including retroviral and lentiviral vectors, for infection or transduction into cells. Alternatively, the construct can be incorporated into vectors capable of episomal replication, e.g. EPV and EBV vectors. The nucleic acid incorporated into the vector can be operatively linked to an expression control sequence such that the expression control sequence controls and regulates the transcription and translation of that polynucleotide sequence.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector can comprise additional elements, for example, the expression vector can have two replication systems, thus allowing it to be maintained in two organisms, for example in human cells for expression and in a prokaryotic host for cloning and amplification.

The term “transfection” as used herein to methods, such as chemical methods, to introduce exogenous nucleic acids, such as the nucleic acid sequences encoding a peptide as described herein into a cell. As used herein, the term transfection does not encompass viral-based methods of introducing exogenous nucleic acids into a cell. Methods of transfection include physical treatments (electroporation, nanoparticles, magnetofection), and chemical-based transfection methods. Chemical-based transfection methods include, but are not limited to those that use cyclodextrin, polymers, liposomes, nanoparticles, cationic lipids or mixtures thereof (e.g., DOPA, Lipofectamine and UptiFectin), and cationic polymers, such as DEAE-dextran or polyethylenimine.

As used herein, the term “viral vector” refers to a nucleic acid vector construct that includes at least one element of viral origin and has the capacity to be packaged into a viral vector particle. The viral vector can contain the nucleic acid encoding a peptide as described herein in place of non-essential viral genes. The vector and/or particle can be utilized for the purpose of transferring any nucleic acids into cells either in vitro or in vivo. Numerous forms of viral vectors are known in the art. The term “replication incompetent” when used in reference to a viral vector means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective (e.g., lacks accessory genes that encode essential proteins for packaging the virus) and viral particles cannot be formed in the patient's cells. The term “transduction” as used herein refers to the use of viral particles or viruses to introduce exogenous nucleic acids into a cell.

Retroviruses, such as lentiviruses, provide a convenient platform for delivery of nucleic acid sequences encoding an agent of interest. A selected nucleic acid sequence can be inserted into a vector and packaged in retroviral particles using techniques known in the art. The recombinant virus can then be isolated and delivered to cells, e.g. in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; which are incorporated by reference herein in their entirety.

In some embodiments, a nucleotide sequence of interest is inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

In some embodiments, a nucleotide sequence encoding a peptide as described herein is inserted into an adeno-associated virus-based expression vector. AAV is a parvovirus which belongs to the genus Dependovirus and has several features not found in other viruses. AAV can infect a wide range of host cells, including non-dividing cells. AAV can infect cells from different species. AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation. AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus. Adeno-associated virus (AAV) has been used with success in gene therapy. AAV has been engineered to deliver genes of interest by deleting the internal nonrepeating portion of the AAV genome (i.e., the rep and cap genes) and inserting a heterologous sequence (in this case, the sequence encoding the agent) between the ITRs. The heterologous sequence is typically functionally linked to a heterologous promoter (constitutive, cell-specific, or inducible) capable of driving expression in the patient's target cells under appropriate conditions. Recombinant AAV virions comprising a nucleic acid sequence encoding an agent of interest can be produced using a variety of art-recognized techniques, as described in U.S. Pat. Nos. 5,139,941; 5,622,856; 5,139,941; 6,001,650; and 6,004,797, the contents of each of which are incorporated by reference herein in their entireties. Vectors and cell lines necessary for preparing helper virus-free rAAV stocks are commercially available as the AAV Helper-Free System (Catalog No. 240071) (Agilent Technologies, Santa Clara, Calif.)

Additional viral vectors useful for delivering nucleic acid molecules encoding a peptide as described herein include those derived from the pox family of viruses, including vaccinia virus and avian poxvirus. Alternatively, avipoxviruses, such as the fowlpox and canarypox viruses, can be used to deliver the genes. The use of avipox vectors in cells of human and other mammalian species is advantageous with regard to safety because members of the avipox genus can only productively replicate in susceptible avian species. Methods for producing recombinant avipoxviruses are known in the art and employ genetic recombination, see, e.g., WO 91/12882; WO 89/03429; and WO 92/03545.

In one aspect, described herein is a cell expressing a vector comprising a nucleic acid encoding the peptides as described herein. In some embodiments, the cell expressing a vector as described herein is a cell suitable for the production of polypeptides. A cell suitable for the production of polypeptides can be a prokaryotic or eukaryotic cell, e.g. bacteria, virus, yeast, fungi, mammalian cells, insect cells, plant cells, and the like. By way of non-limiting example, cells for the production of proteins are commercially available, e.g. bacterial cells (BL21 derived cells—Cat. No. 60401-1, Lucigen; Middleton, Wis. and mammalian cells (293 F cells—Cat. No. 11625-019, Invitrogen; Grand Island, N.Y.).

The peptides of P. acnes hyluronidase or fragments, variants or peptidomimetics thereof (for example, as shown in Table 1) can also be attached to adjuvants. The term “adjuvant” refers to a compound or mixture that enhances the immune response and/or promotes the proper rate of absorption following inoculation, and, as used herein, encompasses any uptake-facilitating agent. Non-limiting examples of adjuvants include, chemokines (e.g., defensins, HCC-1, HCC4, MCP-1, MCP-3, MCP4, MIP-1α, MIP-1β, MIP-1δ, MIP-3a, MIP-2, RANTES); other ligands of chemokine receptors (e.g., CCR1, CCR-2, CCR-5, CCR6, CXCR-1); cytokines (e.g., IL-1β, IL-2, IL-4, IL-5, IL-6, IL-7, IL-8, IL-10, IL-12, IL-13, IL-15, IL-17 (A-F), IL-18; IFNα, IFN-γ; TNF-α; GM-CSF); TGF)-β; FLT-3 ligand; CD40 ligand; other ligands of receptors for those cytokines; Th1 cytokines including, without limitation, IFN-γ, IL-2, IL-12, IL-18, and TNF; Th2 cytokines including, without limitation, IL-4, IL-5, IL-10, and IL-13; and Th17 cytokines including, without limitation, IL-17 (A through F), IL-23, TGF-β and IL-6; immunostimulatory CpG motifs in bacterial DNA or oligonucleotides; derivatives of lipopolysaccharides such as monophosphoryl lipid A (MPL); muramyl dipeptide (MDP) and derivatives thereof (e.g., murabutide, threonyl-MDP, muramyl tripeptide, N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alani-ne-2-(1′-2′-dipalmitoyl-sn-glycero-3hydroxyphosphoryloxy)-ethylamine (CGP 19835A, referred to as MTP-PE)); MF59 (see Int'l Publication No. WO 90/14837); poly[di(carboxylatophenoxy)phosphazene] (PCPP polymer; Virus Research Institute, USA); RIBI (GSK), which contains three components extracted from bacteria, monophosphoryl lipid A, trehalose dimycolate and cell wall skeleton (MPL+TDM+CWS) in a 2% squalene/Tween 80 emulsion; OM-174 (a glucosamine disaccharide related to lipid A; O M Pharma S A, Meyrin, Switzerland); heat shock proteins and derivatives thereof; Leishmania homologs of elF4a and derivatives thereof; bacterial ADP-ribosylating exotoxins and derivatives thereof (e.g., genetic mutants, A and/or B subunit-containing fragments, chemically toxoided versions); chemical conjugates or genetic recombinants containing bacterial ADP-ribosylating exotoxins or derivatives thereof; C3d tandem array; lipid A and derivatives thereof (e.g., monophosphoryl or diphosphoryl lipid A, lipid A analogs, AGP, AS02, ASO4, DC-Chol, Detox, OM-174); ISCOMS and saponins (e.g., Quil A, QS-21, Stimulon® (Cambridge Bioscience, Worcester, Mass.)); squalene; superantigens; or salts (e.g., aluminum hydroxide or phosphate, calcium phosphate). See also Nohria et al. Biotherapy, 7:261-269, 1994; Richards et al., in Vaccine Design, Eds. Powell et al., Plenum Press, 1995; and Pashine et al., Nature Medicine, 11:S63-S68, 4/2005) for other useful adjuvants. Further examples of adjuvants can include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block co-polymer (CytRx, Atlanta Ga.), QS-21 (Cambridge Biotech Inc., Cambridge Mass.), and SAF-M (Chiron, Emeryville Calif.), AMPHIGEN® adjuvant, saponin, Quil A or other saponin fraction, monophosphoryl lipid A, and Avridine lipid-amine adjuvant, and METASTIM®. Other suitable adjuvants can include, for example, surface active substances such as lysolecithin, pluronic polyols, polyanions, peptides, oil or hydrocarbon emulsions, keyhole limpet hemocyanins, dinitrophenol, and others.

In some embodiment, cell may be genetically engineered to express the peptides described herein and the genetically engineered cells may be used for immunotherapy. Examples of cells that may be used include but are not limited to, dendritic cells, T-lymphocytes (T-cells), naïve T cells (T_{N}), memory T cells (for example, central memory T cells (T_{CM}), effector memory cells (T_{EM})), natural killer cells, hematopoietic stem cells and/or pluripotent embryonic/induced stem cells capable of giving rise to therapeutically relevant progeny. In an embodiment, the genetically engineered cells are autologous cells. By way of example, individual T-cells of the invention may be CD4+/CD8−, CD4−/CD8+, CD4−/CD8− or CD4+/CD8+. The T-cells may be a mixed population of CD4+/CD8− and CD4−/CD8+ cells or a population of a single clone. CD4+ T-cells may produce IL-2, IFNγ, TNFα and other T-cell effector cytokines when co-cultured in vitro with cells expressing the peptides (for example CD20+ and/or CD19+ tumor cells). CD8⁺ T-cells may lyse antigen-specific target cells when co-cultured in vitro with the target cells. In some embodiments, T cells may be any one or more of CD45RA⁺ CD62L⁺ naïve cells, CD45RO⁺ CD62L⁺ central memory cells, CD62L⁻ effector memory cells or a combination thereof (Berger et al., Adoptive transfer of virus-specific and tumor-specific T cell immunity. Curr Opin Immunol 2009 21(2)224-232).

In some embodiments, nanoparticles containing the peptide as described herein can be administrated to a subject. In some embodiments, the nanoparticles for use with the peptides described herein may be as described in Levine et al., Polymersomes: A new multi-functional tool for cancer diagnosis and therapy. Methods 2008 Vol 46 pg 25-32 or as described in S Jain, et al., Gold nanoparticles as novel agents for cancer therapy. Br J Radiol. 2012 February; 85(1010): 101-113.

In some embodiments, the cell expressing a vector encoding a peptide as described herein can be a cell of a subject, e.g. a subject administered gene therapy for the treatment, inhibition, reduction of severity and/or slow progression of acne in a subject in need there. Vectors for gene therapy can comprise viral or non-viral vectors as described elsewhere herein.

In some embodiment, the peptides described herein or antigenic variants, fragments or peptidomimetics thereof are linked to macromolecular carriers (such as tetanus toxoid), used in combination with lipid micells or expressed with specific peptide modifications (such as linking to ubiquitin) that target difference immunomodulatory pathways.

### Preparation of Peptides and Vaccines

The peptides described herein and included in the compositions and vaccines described herein are synthesized by any of the techniques known in the art. One technique is through recombinant methods. Another is manual or automated chemical synthesis using individual amino acids, such as solid phase peptide synthesis. Other methods for synthesizing peptides may be readily apparent to one of ordinary skill in the art. One of ordinary skill in the art would be able to determine through routine experimentation which of the immunoreactive peptides are capable of eliciting appropriate antibodies. Non-limiting examples of such methods are described in the examples set forth herein.

Though it is known generally in the art that even single substitutions may have a great impact on immunogenicity of a molecule, due to allelic variants, there are expected to be allowable substitutions within the peptides specifically set forth herein which maintain immunogenicity. That a given substitution results in an immunoreactive peptide can be determined by routine experimentation by making a proposed substitution then testing the immunoreactivity by one of many known assays including those described herein.

In various embodiments, the one or more specific peptide(s) included in the vaccines of the present invention may comprise, consist of, or consist essentially of, all or a portion of SEQ ID NO 1 (Table 1).

As indicated above, one or more of the one or more specific peptides or portion(s) thereof may be modified by any known means of modifying peptides or portions thereof. In some embodiments, one or more of the one or more specific peptides or portions thereof may include one or more amino acid substitutions of any kind known in the art. The use of synthetic and nonstandard amino acids in one or more of the one or more specific peptides or portions thereof is also within the scope of the invention.

A vaccine that includes one or more DNA encoding a portion or all of SEQ ID NO 1. is also within the scope of the present invention, and may further include an appropriate adjuvant and/or carrier described herein.

### Therapeutic Methods

Provided herein are methods for treating, inhibiting, slowing progression of and/or reducing the severity of acne in a subject in need thereof. The methods include providing a pharmaceutical (therapeutic) composition, as described herein, comprising P. acnes hyaluronidase or one or more peptides of P. acnes hyaluronidase and administering an effective amount of the composition to the subject so as to treat, inhibit and/or reduce the severity of acne in the subject. In some embodiments, the peptides are any one or more of the peptides described in Table 1 or fragments, variants or peptidomimetics thereof. In some embodiments, the peptides are combinations of any one or more the peptides described in Table 1. In some embodiments, the methods further comprise administering existing treatments for acne. In some embodiments, the composition and the existing treatments for acne are administered simultaneously. In some embodiments, the composition and the existing therapies are administered sequentially. In some embodiments, the pharmaceutical (therapeutic) composition for use with the methods described herein comprises antibodies that recognize and bind P. acnes hyaluronidase. In some embodiments, the antibody binds one or more epitopes having the sequence set forth in Table 1. The antibodies may be monoclonal or polyclonal.

Also provided herein are methods for treating, inhibiting, slowing progression of and/or reducing the severity of an acne-induced condition in a subject in need thereof. The methods include providing a pharmaceutical (therapeutic) composition, as described herein, comprising P. acnes hyaluronidase or one or more peptides of P. acnes hyaluronidase and administering an effective amount of the composition to the subject so as to treat, inhibit, slow progression of and/or reduce the severity of an acne-induced condition in the subject. In some embodiments, the peptides are any one or more of the peptides described in Table 1 or fragments, variants or peptidomimetics thereof. In some embodiments, the peptides are combinations of any one or more the peptides described in Table 1. In some embodiments, the methods further comprise administering existing treatments for acne. In some embodiments, the composition and the existing treatments for acne are administered simultaneously. In some embodiments, the composition and the existing therapies are administered sequentially. In some embodiments, the pharmaceutical (therapeutic) composition for use with the methods described herein comprises antibodies that recognize and bind P. acnes hyaluronidase. In some embodiments, the antibody binds one or more epitopes having the sequence set forth in Table 1. The antibodies may be monoclonal or polyclonal.

Also provided herein are methods for preventing acne in a subject in need thereof. The methods include providing a vaccine, as described herein, comprising P. acnes hyaluronidase or one or more peptides of P. acnes hyaluronidase and administering an effective amount of the vaccine to the subject so as to prevent acne in the subject. In some embodiments, the acne-induced condition is inflammation, an abscess, pain or combinations thereof. In some embodiments, the peptides are any one or more of the peptides described in Table 1 or fragments, variants or peptidomimetics thereof. In some embodiments, the peptides are combinations of any one or more the peptides described in Table 1. In some embodiments, the methods further comprise administering existing treatments for acne. In some embodiments, the vaccine and the existing treatments for acne are administered simultaneously. In some embodiments, the vaccine and the existing therapies are administered sequentially.

In various embodiments, the compositions disclosed herein result in a bacterial hyaluronidase-specific immune response through treatment or immunization with P. acnes hyaluronidase or any one or more peptides comprising, consisting of or consisting essentially of the sequences set forth in Table 1 or fragments, variants or peptidomimetics thereof.

, In some embodiments, the immunization includes administering to the subject one or more portions or all of the P. acnes hyaluronidase, or analogs thereof. In various embodiments, the inventive compositions and methods described herein may lead to prevention, improvement or elimination of acne and/or related conditions associated with inflammation. There may also be immunologic protection against re-activation of P. acnes infection on the skin. Combination therapies, in which a vaccine is administered in conjunction with any traditional treatment described or referenced herein is also within the scope of the invention.

In some embodiments, a therapy disclosed herein provides sustained immunologic protection against reactivation of P. acnes infection and associated conditions, and reduces the need for traditional skin treatments.

Some advantages of vaccine therapies disclosed herein in various embodiments include: 1) definitive treatment of P. acnes infection and associated conditions; 2) subsequent protection against the development of P. acnes infection and associated conditions; and 3) reduction in the severity of P. acnes infection and associated conditions.

In various embodiments, the present invention provides a method of treating, preventing, reducing the likelihood of having, reducing the severity of and/or slowing the progression of a condition in a subject. The method may consist of or may consist essentially of or may comprise: providing a vaccine targeting P. acnes hyaluronidase; and administering a therapeutically effective amount of the vaccine to the subject, thereby treating, preventing, reducing the likelihood of having, reducing the severity of and/or slowing the progression of the condition in the subject.

In various embodiments, the condition may include but is in no way limited to acne, associated inflammation, pain, secondary infection, and related conditions.

In various embodiments, the subject is a human. In some embodiments, the subject is a male human. In other embodiments, the subject is a female human. In various embodiments, the subject is a mammalian subject including but not limited to human, monkey, ape, dog, cat, cow, horse, goat, pig, rabbit, mouse and rat.

In various embodiments, one or more traditional acne treatment and the P. acnes hyaluronidase targeting vaccine are administered concurrently, sequentially, or alternatively. In various embodiments, the P. acnes hyaluronidase targeting vaccine is administered before, during or after administering one or more traditional acne treatment.

In accordance with certain embodiments of the invention, pharmaceutical compositions and/or vaccines described herein may be administered using the appropriate modes of administration. In accordance with the invention, various routes of administration may be utilized with the methods described herein, including but not limited to intravenous, intraarterial, intramuscular, subcutaneous, intraperitoneal, aerosol, nasal, via inhalation, oral, transmucosal, transdermal, parenteral, implantable pump or reservoir, continuous infusion, enteral application, topical application, local application, capsules and/or injections. In various embodiments, the P. acnes hyaluronidase targeting vaccine is administered epidurally, intradurally, topically, intravascularly, intravenously, intraarterially, intramuscularly, subcutaneously, intraperitoneally, intranasally, or orally.

Typical dosages of an effective amount of the pharmaceutical compositions and/or vaccines can be in the ranges indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

In various embodiments, the effective amount of the pharmaceutical compositions and/or vaccines is any one or more of about 0.001-0.01 μg/kg, 0.01-0.1 μg/kg, 0.1-0.5 μg/kg, 0.5-5 μg/kg, 5-10 μg/kg, 10-20 μg/kg, 20-50 μg/kg, 50-100 μg/kg, 100-200 μg/kg, 200-300 μg/kg, 300-400 μg/kg, 400-500 μg/kg, 500-600 μg/kg, 600-700 μg/kg, 700-800 μg/kg, 800-900 μg/kg, or 900-1000 μg/kg or combinations thereof.

In various embodiments, the pharmaceutical composition or vaccine is administered once, twice, three or more times. In certain embodiments, the pharmaceutical composition or vaccine is administered to a human. In some embodiments, the pharmaceutical composition or vaccine is administered to a teenage human. The optimum dosages and frequencies will be apparent to a person of skill in the art.

In some embodiments, the P. acnes hyaluronidase vaccine may be administered at the prevention stage of a condition (i.e., when the subject has not developed the condition but is likely to or in the process to develop the condition). In other embodiments, the P. acnes hyaluronidase vaccine may be administered at the treatment stage of a condition (i.e., when the subject has already developed the condition). As non-limiting examples, the target condition is P. acnes infection, acne, or a related condition. In exemplary situations, the patient may be treated with the methods described herein when the patient has not yet developed acne, or is likely to develop acne, or is in the process of developing acne, or has already developed acne.

In various embodiments, the present invention provides a composition that may consist of or may consist essentially of or may comprise P. acnes hyaluronidase vaccine.

In accordance with the present invention, the composition may be used for treating, preventing, reducing the likelihood of having, reducing the severity of and/or slowing the progression of a condition in a subject. In various embodiments, the condition is a P. acnes infection, acne, a secondary infection, a related condition, or combinations thereof.

In various embodiments, the P. acnes hyaluronidase peptides described herein in the pharmaceutical compositions or vaccines are provided in about 1-10, 10-20, 20-30, 30-40, 40-50, 50-60, 60-70, 70-80, 80-90, or 90-100 μg per dose. In various embodiments, the P. acnes hyaluronidase peptides described herein in the pharmaceutical compositions or vaccines are provided in μg per kilogram body weight of the subject, for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/kg.

In various embodiments, the composition is formulated for epidural, intradural, topical, intravascular, intravenous, intraarterial, intramuscular, subcutaneous, intraperitoneal, intranasal or oral administration. In one embodiment, a composition comprising a P. acnes hyaluronidase vaccine is formulated for intramuscular administration. Preferred compositions will also exhibit minimal toxicity when administered to a mammal.

### Pharmaceutical Compositions

Provided herein are pharmaceutical (therapeutic) compositions and vaccines comprising, consisting of or consisting essentially of P. acnes hyluronidase or peptides of P. acnes hyluronidase or fragments, derivatives, variants or peptidomimetics thereof, and a pharmaceutically acceptable excipient. Exemplary embodiments of peptides of P. acnes hyluronidase are shown in Table 1 and have the sequences set forth in SEQ ID NOs: 2-29. In various embodiments, the pharmaceutical compositions described herein are used to treat, inhibit, reduce the severity of and/or slow progression of acne in a subject in need thereof. In some embodiments, the vaccine described herein is used to prevent acne in a subject in need thereof.

Also provided herein are pharmaceutical compositions comprising antibodies that recognize and bind P. acnes hyluronidase or fragments, derivatives, variants or peptidomimetics thereof (for examples, Table 1), and a pharmaceutically acceptable excipient. In various embodiments, the pharmaceutical compositions comprising the antibodies described herein are used to treat, inhibit, reduce the severity of and/or slow progression of acne in a subject in need thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. In some embodiments, the pharmaceutical composition may be administered orally. In some embodiments, the pharmaceutical composition may be administered to the colon by rectal suppository or enema. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of therapeutic agents that increase an immune response in the subject other than directly into a target site, tissue, or organ, such that it enters the subject's circulatory system and, thus, is subject to metabolism and other like processes.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

The therapeutic agents described herein may be administered to the patient in a single dose or in multiple doses. When multiple doses are administered, the doses may be separated from one another by, for example, one hour, three hours, six hours, eight hours, one day, two days, one week, two weeks, or one month. For example, the therapeutic may be administered for, e.g., 2, 3, 4, 5, 6, 7, 8, 10, 15, 20, or more weeks. In various embodiments, the composition is administrated to the subject 1-3 times per day or 1-7 times per week. In various embodiments, the composition is administrated to the subject for 1-5 days, 1-5 weeks, 1-5 months, or 1-5 years. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity. While the attending physician ultimately will decide the appropriate amount and dosage regimen, therapeutically effective amounts of the one or more peptides as disclosed herein or a mutant, variant, analog or derivative thereof may be provided at a dose of 0.0001, 0.01, 0.01 0.1, 1, 5, 10, 25, 50, 100, 500, or 1,000 mg/kg or μg/kg. Effective doses may be extrapolated from dose-response curves derived from in vitro or animal model test bioassays or systems.

An effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the therapeutic agents that increase the immune response, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” or “pharmaceutically acceptable excipient” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, therapeutic agents that increase an immune response. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (18) pH buffered solutions; (19) polyesters, polycarbonates and/or polyanhydrides; (20) bulking agents, such as polypeptides and amino acids (21) serum components, such as serum albumin, HDL and LDL; (22) C2-C12 alchols, such as ethanol; and (23) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The pharmaceutical compositions and/or vaccines described herein that induce or increase an immune response described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; (7) enterically or (8) nasally. Additionally, the pharmaceutical compositions and/or vaccines described herein can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 3,270,960.

Further embodiments of the formulations and modes of administration of the pharmaceutical composition and vaccines described herein that can be used in the methods described herein are illustrated below.

Parenteral Dosage Forms. Parenteral dosage forms of pharmaceutical compositions and/or vaccines described herein can also be administered to a subject by various routes, including, but not limited to, subcutaneous, intravenous (including bolus injection), intramuscular, and intra-arterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol formulations. Pharmaceutical compositions and/or vaccines described herein can be packaged in a pressurized aerosol container together with suitable propellants, for example, hydrocarbon propellants like propane, butane, or isobutane with conventional adjuvants. Pharmaceutical compositions and/or vaccines described herein can also be administered in a non-pressurized form such as in a nebulizer or atomizer. Pharmaceutical compositions and/or vaccines described herein can also be administered directly to the airways in the form of a dry powder, for example, by use of an inhaler.

Suitable powder compositions include, by way of illustration, powdered preparations of the pharmaceutical compositions and/or vaccines described herein thoroughly intermixed with lactose, or other inert powders acceptable for intra-bronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the pharmaceutical compositions and/or vaccines described herein further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms. In some embodiments of the methods described herein, the pharmaceutical compositions and/or vaccines described herein can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound's onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the pharmaceutical compositions and vaccines described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

In some embodiments, the pharmaceutical compositions and/or vaccines described herein for use in the methods described herein are administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of the pharmaceutical compositions and/or vaccines described herein administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### Kits of the Invention

In various embodiments, the present invention provides a kit for treating, preventing, reducing the severity of and/or slowing the progression of a condition in a subject. The kit may consist of or may consist essentially of or may comprise: a P. acnes hyaluronidase vaccine to treat, prevent, reduce the likelihood of having, reduce the severity of and/or slow the progression of the condition in the subject.

The kit is an assemblage of materials or components, including at least one of the inventive compositions or components. The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, spray bottles or cans, diluents, buffers, pharmaceutically acceptable carriers, syringes, catheters, applicators (for example, applicators of cream, gel or lotion etc.), pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the compositions or components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in assays and therapies. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### EXAMPLES

The invention will be further explained by the following Examples, which are intended to be purely exemplary of the invention, and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Therapeutic Strategies

Development of Pharmacologic Blockers of P. acnes hyase:

P. acnes hyase shares little homology to hyase from other gram-positive pathogens. There are no pharmacological agents available that block the P. acnes enzyme. Therefore some embodiments of the invention include peptide and small molecule drugs developed using structure-based approaches which involve the expression of recombinant hyase, and development hits, X-ray crystallography and structure-activity relationship studies.

Expression of Hyase from P. acnes:

In development are recombinant P. acnes hyase protein, a full length protein and an enzyme active site, using a PET28 expression vector. Both proteins can be used for crystallography and for vaccine experiments described below.

Peptide Based Drug Discovery:

To identify peptide ligands, the standard phage display technique can be utilized. Briefly, Phage libraries either PHD7 and PHD12 (New England Biolab) that display either 7- or 12-residue peptides, a random 12-mer library, or random 11-residue peptides are fused to gene III in which the central residue of each library is fixed with a different residue. Each library will have a complexity between 5×10⁸ and 2×10⁹. Using the recombinant hyase protein as a target, affinity selection is performed using phage ELISA. To measure specificity, one of the Gram-positive hyases is used as a target. High affinity peptides displayed by phage are sequenced. Peptides are synthesized using a vendor and are tested for affinity and biological activity using isothermal titration calorimetry (ITC). Hyase inhibitory activity is measured.

X-Ray Crystallography:

To facilitate identification of small molecule inhibitors, the structure of P. acnes hyase is determined. Briefly, several crystallization screens using robotics, and suitable crystals are used to collect diffraction data followed by structure determination using molecular replacement technique. The co-complex structure with the peptide can provide insight into the binding mode of the inhibitor. To develop small molecule inhibitors, a two-fold approach can be used: (a) Fragment-based drug discovery (FBDD). The inventors have obtained a collection of 250 chemical fragments tailored for FBDD. Fragments are first screened using either thermal shift or by surface plasmon resonance. Hits identified from SPR/thermal shift are co-crystallized and its binding location can be resolved using X-ray crystallography. A set of fragments identified from the three-dimensional structure can be further developed into a library of compounds using AMRI <wwwdotamriglobaldotcom>. The library compounds are screened using SPR/thermal shift and biological assay in an iterative manner to identify a lead drug. (b) A virtual screening approach (Glide, Schrodinger, Inc) can be used to screen a Zinc database to identify potential inhibitors. Hits are identified.

Development of a Vaccine Against P. acnes hyase:

The crystal structure of P. acnes hyase can be used to identify antigenic peptides, as described herein. These peptides and purified P. acnes hyase can be used to generate antibodies, and screened for activity, as described herein.

In vivo testing: Inhibitors or vaccine candidates developed as described above can be tested in a murine P. acnes infection model. Additionally, inflammation may be blocked in the acne model using GBS hyase to degrade HA fragments and diHA to block TLR2/4. This approach has been successfully applied to block inflammation in an acute lung injury model induced by LPS.

P. acnes induced inflammation has also been implicated in prostate cancer and sarcoidosis, and therefore the inhibitors and vaccines described herein may be useful for those indications as well.

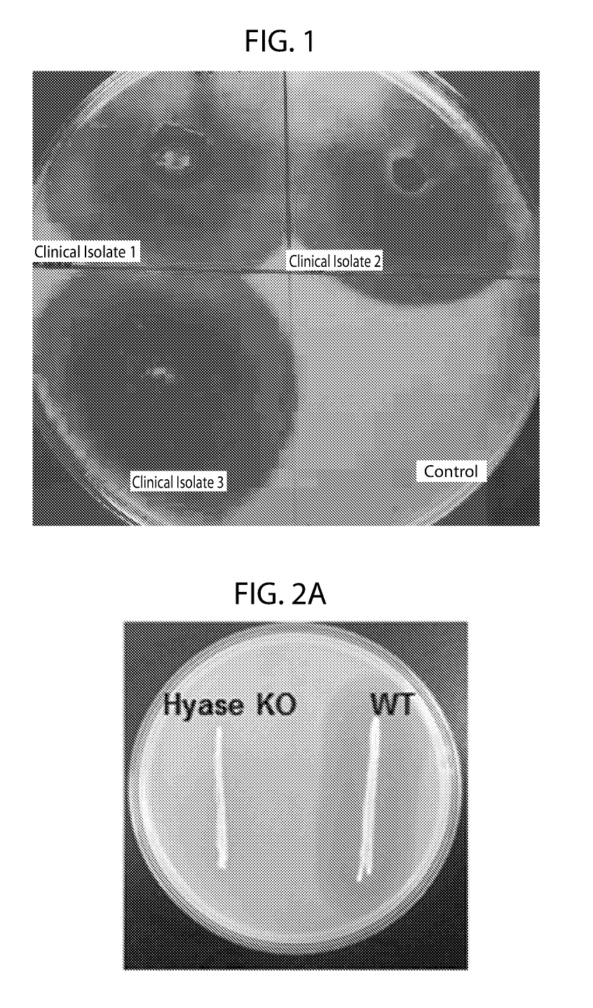

As shown in FIG. 1, three clinical isolates and one control strain of P. acnes were placed on an agar plate containing hyaluronan and incubated overnight. A clearing zone is observed. The zone of clearing is indicative of hyaluronidase activity. All three clinical isolates secrete hyaluronidase.

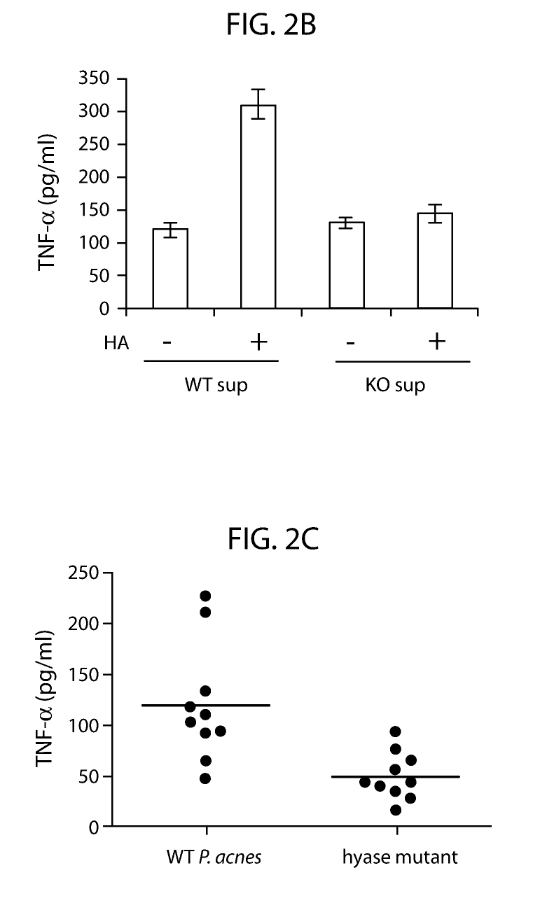

As showin in FIG. 2 (a) P. acnes WT and hyaluronidase mutant were placed on a on a HA plate and incubated overnight. A zone of clearing shows the WT strain secretes hyaluronidase while the mutant strain does not. As shown in FIG. 2b, supernatants from WT or hyaluronidase mutant were used to digest hyaluronan. Hyaluronan alone or the resulting digested fragments were used to stimulate BMDM for 5 hours and the amount of secreted TNF-α was then quantified. This shows that the WT strain produces stimulatory HA fragments while the mutant does not affect HA since it is missing hyaluronidase. As shown in FIG. 2c, mice were infected with P. acnes WT or hyaluronidase mutant. Twenty-four hours following infection, the skin was harvested and TNF-α measured. The mutant produces less TNF-α supporting the importance of the enzyme on inflammation.

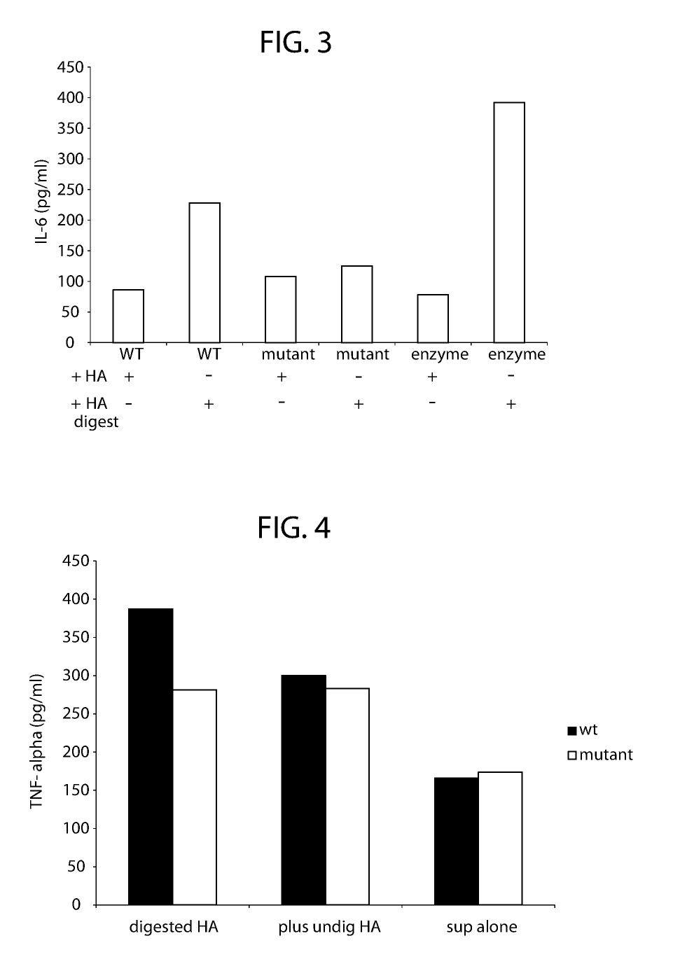

FIG. 3 depicts the effect of HA digest by P. acnes hyaluronidase on pro-inflammatory cytokine release from macrophages. The WT, mutant, and purified enzyme were used to digest hyaluronan. Undigested or digested hyaluronan was used to stimulate macrophages for 5 hours and the pro-inflammatory cytokine IL-6 was measured.

FIG. 4 illustrates the effect of HA digest by P. acnes hyaluronidase on pro-inflammatory cytokine release from a human keratinocyte (HaCaT) cell line. P. acnes WT or mutant supernatant was mixed with hyaluronan. The supernatant of each alone, with undigested hyaluronan, or with the resulting digested fragments was used to stimulate human keratinocytes for 12 hours and TNF-α produced was determined.

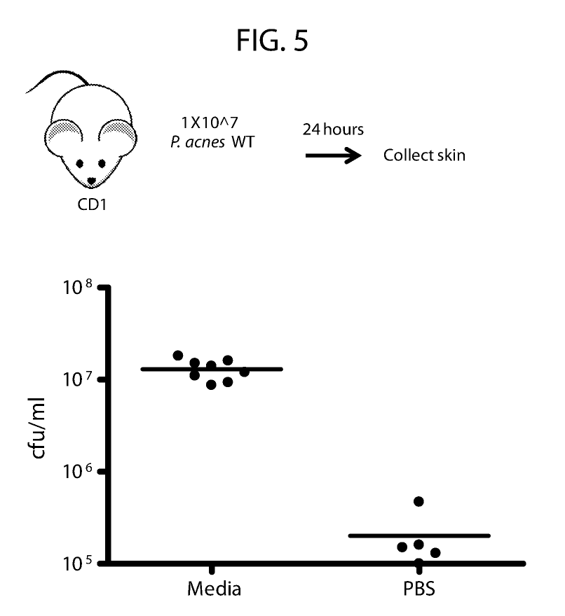

FIG. 5 depicts a new mouse model of P. acnes that was generated for therapeutic testing. CD1 mice were infected with P. acnes resuspended in either media or PBS. After 24 hours, skin was collected from infected areas, homogenized, and the cfu determined. P. acnes in PBS is not stable in a mouse model of infection where P. acnes in media is.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method of treating, inhibiting, reducing the likelihood of having, reducing the severity of and/or slowing the progression of a condition in a subject, comprising:
providing a composition comprising P. acnes hyluronidase or peptides of P. acnes hyluronidase or fragments, derivatives, variants or peptidomimetics thereof; and
administering a therapeutically effective amount of the composition to the subject, thereby treating, reducing the likelihood of having, reducing the severity of and/or slowing the progression of the condition in the subject.

2. The method of claim 1, wherein the condition is acne, an infection secondary to acne, or an acne-induced condition.

3. The method of claim 2, wherein the acne-induced condition is inflammation, an abscess, pain, or combinations thereof.

4. The method of claim 1, wherein the subject is a human.

5. The method of claim 1, wherein the subject is a male human.

6. The method of claim 1, wherein the subject is a female human.

7. The method of claim 1, wherein the composition is administered simultaneously with a topical acne treatment, or subsequent to a topical acne treatment, or prior to a topical acne treatment, or any combination thereof.

8. The method of claim 1, wherein the composition comprises any one or more of the peptides having the sequence set forth in SEQ ID NOs: 2-29.

9. A pharmaceutical composition comprising one or more peptides having the sequence set forth in SEQ ID NOs. 2-29 and a pharmaceutically acceptable carrier.

10. The pharmaceutical composition of claim 9, further comprising an adjuvant.

11. A pharmaceutical composition comprising one or more antibodies that specifically binds to an antigen having the sequence set forth in any one or more of SEQ ID NOs. 1-29.

12. The composition of claim 11, wherein the antibody is a monoclonal antibody.

13. The composition of claim 11, wherein the antibody is a polyclonal antibody.

14. A polynucleotide encoding a fragment of SEQ ID NO. 1.

15. The polynucleotide of claim 14, wherein the fragment comprises the polynucleotide sequence encoding any one of the peptides in SEQ ID NOs.: 2-29.

16. A polypeptide encoded by the polynucleotide of claim 15.

17. A cDNA molecule of claim 15.

18. A vector comprising the cDNA molecule of claim 17.

19. A host-vector system comprising the vector of claim 18, transfected into a compatible host cell.

20. The host-vector system of claim 19, wherein the compatible host cell is a prokaryotic cell or a eukaryotic cell.

21. A vaccine comprising the polynucleotide of claim 15.

22. A vaccine comprising the polypeptide of claim 16.

23. A kit for treating, reducing the likelihood of having, reducing the severity of and/or slowing the progression of a condition in a subject in need thereof, comprising:
a composition comprising P. acnes hyluronidase or peptides of P. acnes hyluronidase or fragments, derivatives, variants or peptidomimetics thereof; and
instructions for using the composition to treat, reduce the likelihood of having, reduce the severity of and/or slow the progression of the condition in the subject.

24-28. (canceled)

29. The kit of claim 23, further comprising a topical acne treatment.


SEQUENCE LISTING (29 sequences)

SEQ ID NO: 1 - protein, 813 aa, Propionibacterium acnes
MFGTPSRRTFLTASALSAMALAASPTVTDAIAAPGPDSWSALCERWIDIITGRRAARTSDPRARAIIAKTDRKVAEILTDLVSGSSRQTVLISADLRKEQSPFITKTARAIESMACAWATPGSSYHKDPEILSACIEGLRDFCRLRYNPSQDEYGNWWDWEDGASRAVADVMCILHDVLPPEVMSAAAAGIDHFIPDPWFQQPASVKPTANPVQPVVSTGANRMDLTRAVMCRSIATGDEKRLRHAVDGLPDAWRVTTEGDGFRADGGFIQHSHIPYTGGYGDVLFSGLAMLFPLVSGMRFDIVESARKAFHDQVERGFIPVMYNGQILDDVRGRSISRINESAAMHGISIARAMLMMADALPTHRAEQWRGIVHGWMARNTFDHLSEPSTLVDISLFDAAAKARPVPESSTPSYFASMDRLVHRTADWLITVSNCSDRIAWYEYGNGENEWASRTSQGMRYLLLPGDMGQYEDGYWATVDYSAPTGTTVDSTPLKRAVGASWAAKTPTNEWSGGLASGSWSAAASHITSQDSALKARRLWVGLKDAMVELTTDVTTDASRAITVVEHRKVASSSTKLLVDGNRVSSATSFQNPRWAHLDGVGGYVFATDTDLSADVATRKGTWIDVNPSRKVKGADEVIERAYASLHVTHHDRPVAWALLPTASRSHTMALATRPGVEPFTVLRNDATVQAVRSAGALLTKDPTVVTTLAFWKPATCGGVAVNRPALVQTRESANQMEVVIVEPTQKRGSLTVTIEGSWKVKTADSHVDVSCENAAGTLHVDTAGLGGQSVRVTLARQVTQTPSGGGRHDRA

SEQ ID NO: 2 - protein, 25 aa, Artificial Sequence
  synthetic construct
FLTASALSAMALAASPTVTDAIAAP

SEQ ID NO: 3 - protein, 8 aa, Artificial Sequence
  synthetic construct
SWSALCER

SEQ ID NO: 4 - protein, 22 aa, Artificial Sequence
  synthetic construct
KVAEILTDLVSGSSRQTVLISA

SEQ ID NO: 5 - protein, 7 aa, Artificial Sequence
  synthetic construct
ESMACAW

SEQ ID NO: 6 - protein, 21 aa, Artificial Sequence
  synthetic construct
KDPEILSACIEGLRDFCRLRY

SEQ ID NO: 7 - protein, 55 aa, Artificial Sequence
  synthetic construct
ASRAVADVMCILHDVLPPEVMSAAAAGIDHFIPDPWFQQPASVKPTANPVQPVVS

SEQ ID NO: 8 - protein, 9 aa, Artificial Sequence
  synthetic construct
LTRAVMCRS

SEQ ID NO: 9 - protein, 7 aa, Artificial Sequence
  synthetic construct
LRHAVDG

SEQ ID NO: 10 - protein, 9 aa, Artificial Sequence
  synthetic construct
FIQHSHIPY

SEQ ID NO: 11 - protein, 26 aa, Artificial Sequence
  synthetic construct
YGDVLFSGLAMLFPLVSGMRFDIVES

SEQ ID NO: 12 - protein, 11 aa, Artificial Sequence
  synthetic construct
ERGFIPVMYNG

SEQ ID NO: 13 - protein, 9 aa, Artificial Sequence
  synthetic construct
AMHGISIAR

SEQ ID NO: 14 - protein, 7 aa, Artificial Sequence
  synthetic construct
WRGIVHG

SEQ ID NO: 15 - protein, 27 aa, Artificial Sequence
  synthetic construct
FDHLSEPSTLVDISLFDAAAKARPVPE

SEQ ID NO: 16 - protein, 21 aa, Artificial Sequence
  synthetic construct
MDRLVHRTADWLITVSNCSDR

SEQ ID NO: 17 - protein, 8 aa, Artificial Sequence
  synthetic construct
GMRYLLLP

SEQ ID NO: 18 - protein, 7 aa, Artificial Sequence
  synthetic construct
YWATVDY

SEQ ID NO: 19 - protein, 8 aa, Artificial Sequence
  synthetic construct
PLKRAVGA

SEQ ID NO: 20 - protein, 28 aa, Artificial Sequence
  synthetic construct
SAAASHITSQDSALKARRLWVGLKDAMV

SEQ ID NO: 21 - protein, 22 aa, Artificial Sequence
  synthetic construct
RAITVVEHRKVASSSTKLLVDG

SEQ ID NO: 22 - protein, 11 aa, Artificial Sequence
  synthetic construct
HLDGVGGYVFA

SEQ ID NO: 23 - protein, 30 aa, Artificial Sequence
  synthetic construct
GADEVIERAYASLHVTHHDRPVAWALLPTA

SEQ ID NO: 24 - protein, 15 aa, Artificial Sequence
  synthetic construct
LATRPGVEPFTVLRN

SEQ ID NO: 25 - protein, 44 aa, Artificial Sequence
  synthetic construct
ATVQAVRSAGALLTKDPTVVTTLAFWKPATCGGVAVNRPALVQT

SEQ ID NO: 26 - protein, 9 aa, Artificial Sequence
  synthetic construct
QMEVVIVEP

SEQ ID NO: 27 - protein, 7 aa, Artificial Sequence
  synthetic construct
GSLTVTI

SEQ ID NO: 28 - protein, 22 aa, Artificial Sequence
  synthetic construct
KTADSHVDVSCENAAGTLHVDT

SEQ ID NO: 29 - protein, 15 aa, Artificial Sequence
  synthetic construct
GGQSVRVTLARQVTQ